(19) United States
(12) Patent Application Publication
(10) Pub. No.: US 2014/0025069 A1
(43) Pub. Date: Jan. 23, 2014

(54) RENAL NERVE MODULATION CATHETER DESIGN

(71) Applicant: BOSTON SCIENTIFIC SCIMED, INC., MAPLE GROVE, MN (US)

(72) Inventors: MARTIN R. WILLARD, BURNSVILLE, MN (US); JASON P. HILL, BROOKLYN PARK, MN (US)

(21) Appl. No.: 13/944,707

(22) Filed: Jul. 17, 2013

Related U.S. Application Data
(60) Provisional application No. 61/672,673, filed on Jul. 17, 2012.
(60) Provisional application No. 61/694,074, filed on Aug. 28, 2012.

(51) Int. Cl. A61B 18/14

(52) U.S. Cl. CPC A61B 18/1492
USPC 606/41

(57) ABSTRACT

Systems for nerve and tissue modulation are disclosed. An example system may an elongate shaft including an expandable frame coupled to the shaft adjacent to a distal end of the shaft. The frame may include a plurality of electrically conductive regions for emitting an electrical current comprising at least a first electrically conductive region and a second electrically conductive region. The system may further include a ground pad and a control unit electrically coupled to the plurality of electrically conductive regions and the ground pad. The control and power unit is configured to operate in unipolar mode and bipolar mode during the same procedure.

### CROSS-REFERENCE TO RELATED APPLICATIONS

This application claims priority under 35 U.S.C. §119 to U.S. Provisional Application Ser. No. 61/672,673, filed Jul. 17, 2012; and to U.S. Provisional Application Ser. No. 61/694,074, filed Aug. 28, 2012, all of which are herein incorporated by reference.

### TECHNICAL FIELD

The present invention relates to methods and apparatuses for nerve modulation techniques such as ablation of nerve tissue or other destructive modulation technique through the walls of blood vessels and monitoring thereof.

### BACKGROUND

Certain treatments require the temporary or permanent interruption or modification of select nerve function. One example treatment is renal nerve ablation which is sometimes used to treat hypertension and other conditions related to hypertension and congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular renal nerves using a radio frequency (RF) electrode in an off-wall configuration or in a configuration in contact with the vessel wall. RF electrodes may ablate the perivascular nerves, but may also damage the vessel wall as well. Control of the ablation may effective ablate the nerves while minimizing injury to the vessel wall. Sensing electrodes may allow the use of impedance measuring to monitor tissue changes. It is therefore desirable to provide for alternative systems and methods for intravascular nerve modulation.

### SUMMARY

The disclosure is directed to several alternative designs, materials and methods of manufacturing medical device structures and assemblies for performing and monitoring tissue changes.

Accordingly, one illustrative embodiment is a system for nerve modulation that may include an elongate member having a distal end region and a plurality of electrodes at the distal end region, at least one return electrode patch; and a control and power unit electrically connected to the plurality of electrodes and the return electrode patch, wherein the control and power unit is configured to operate in unipolar mode and bipolar mode during the same procedure.

The control and power unit may be configured to operate in unipolar mode while operating in bipolar mode, may be configured to switch between unipolar mode and bipolar mode upon receiving user input, or may be configured to periodically switch between unipolar and bipolar mode. In other embodiments, the control and power unit may be configured to switch between a first mode where the system operates in unipolar and bipolar mode simultaneously and a second mode where the system operates in unipolar mode only, or between a first mode where the system operates in unipolar and bipolar mode simultaneously and a second mode where the system operates in bipolar mode only, or between a first mode where the system operates in unipolar and bipolar mode simultaneously, a second mode where the system operates in unipolar mode only and a third mode where the system operates in bipolar mode only.

Another example system may include a tubular member having a proximal end region, a distal end region, and a lumen extending therebetween. An elongate shaft may be slidably disposed within the lumen of the tubular member. An expandable frame may be coupled to the shaft adjacent to a distal end of the shaft. A first set of electrodes including one or more electrodes may be disposed adjacent a proximal end of the expandable frame. A second set of electrodes including one or more electrodes may be disposed adjacent a distal end of the expandable frame. A control unit electrically may be coupled to the first set of electrodes and the second set of electrodes. The system may also include a ground pad. The first set of electrodes, the second set of electrodes, and the ground pad may be electrically connected to the control unit.

Another example system for nerve modulation may include an elongate shaft having a proximal end region, a distal end region, and a lumen disposed therebetween. An actuation element may be slidably disposed within the lumen of the elongate shaft. An expandable frame may be coupled to a distal end region of the actuation element. The expandable frame may have a proximal end and a distal end. A first set of electrodes may be disposed adjacent the proximal end of the expandable frame. A second set of electrodes may be disposed adjacent the distal end of the expandable frame.

Another example system for nerve modulation may include an elongate shaft having a proximal end region, a distal end region, and a lumen disposed therebetween. An expandable positioning element may be slidably disposed within the lumen of the elongate shaft. The expandable positioning element may have a proximal end and a distal end. A first set of nerve modulation elements comprising at least one nerve modulation element may be disposed adjacent the proximal end of the positioning element. A second set of nerve modulation elements comprising at least one nerve modulation element may be disposed adjacent the distal end of the positioning element.

Another example system for nerve modulation may include an elongate shaft having a proximal end and a distal end. An expandable member may be coupled to the elongate shaft adjacent to the distal end of the shaft. The system may further include a plurality of electrically conductive regions disposed on the expandable member for emitting an electrical current. The plurality of electrically conductive regions may comprise at least a first electrically conductive region and a second electrically conductive region. The system may further include a ground pad and a control unit electrically coupled to the plurality of electrically conductive regions and the ground pad. The control and power unit may be configured to operate in unipolar mode and bipolar mode during the same procedure.

Another example system for nerve modulation may include an elongate shaft having a proximal end region, a distal end region, and a lumen disposed therebetween. An actuation element may be slidably disposed within the lumen of the elongate shaft. The system may further include an expandable basket including two or more struts and having a proximal end and a distal end. The expandable frame may be coupled to a distal end region of the actuation element. The system may further include a return electrode patch. A first electrically conductive region may be disposed on a first strut of the two or more struts and a second electrically conductive region may be disposed on a second strut of the two or more struts.

Embodiments also pertain to methods of using such systems.

The above summary of some example embodiments is not intended to describe each disclosed embodiment or every implementation of the invention.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 illustrates a distal end of an illustrative renal nerve modulation system in situ.

FIG. 3 illustrates a distal end of an illustrative renal nerve modulation system.

FIG. 4 illustrates a distal end of an illustrative renal nerve modulation system.

FIG. 5 illustrates a schematic view of an illustrative renal nerve modulation system according to embodiments of the present disclosure.

FIG. 6 illustrates is a schematic view illustrating a collapsed state of the renal nerve modulation system according to embodiments of the present disclosure.

FIG. 7 illustrates a schematic view illustrating the renal nerve modulation system shown in FIG. 5, and a method of using the system according to embodiments of the present disclosure.

FIG. 8 illustrates a distal end region of another illustrative renal nerve modulation system.

FIG. 9 illustrates a distal end region of another illustrative renal nerve modulation system.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Many of the devices and methods are disclosed herein in the context of renal nerve modulation through a blood vessel wall. However, devices and methods of other embodiments may be used in other contexts, such as applications other than where nerve modulation and/or ablation are desired. It is contemplated that the devices and methods may be used in other treatment locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pulmonary vein isolation, tumor ablation, benign prostatic hyperplasia therapy, nerve excitation or blocking or ablation, modulation of muscle activity, hyperthermia or other warming of tissues, etc. The disclosed methods and apparatus can be applied to any relevant medical procedure, involving both human and non-human subjects.

In some instances, it may be desirable to ablate perivascular renal nerves with deep target tissue heating. As energy passes from a modulation element to the desired treatment region the energy may heat both the tissue and the intervening fluid (e.g. blood) as it passes. As more energy is used, higher temperatures in the desired treatment region may be achieved thus resulting in a deeper lesion. Monitoring tissue properties may, for example, verify effective ablation, improve safety, and optimize treatment time. The term modulation refers to ablation and other techniques that may alter the function of affected nerves and other tissue.

In some instances, impedance monitoring may be used to detect changes in target tissues as ablation progresses. Sensing electrodes may be provided in addition to the modulation element. In some instances, the impedance may not be directly measured, but may be a function of the current distribution between the sensing electrodes. In general, the resistance of the surrounding tissue may decrease as the temperature of the tissue increases until a point where the tissue begins to denature or irreversibly change, for example, at approximately 50-60° C. Once the tissue has begun to denature the resistance of the tissue may increase. As the target tissue is ablated, the change in impedance may be analyzed to determine how much tissue has been ablated. The power level and duration of the ablation may be adjusted accordingly based on the impedance of the tissue. In some instances, overall circuit impedance may be monitored and modulation systems may utilize a standard power delivery level, but variation in local tissue impedance can cause unpredictable variation in the ablation effect on the target tissue and in local artery wall heating. It may be desirable to provide a simple way to determine local tissue impedance in order to control ablation using a split electrode.

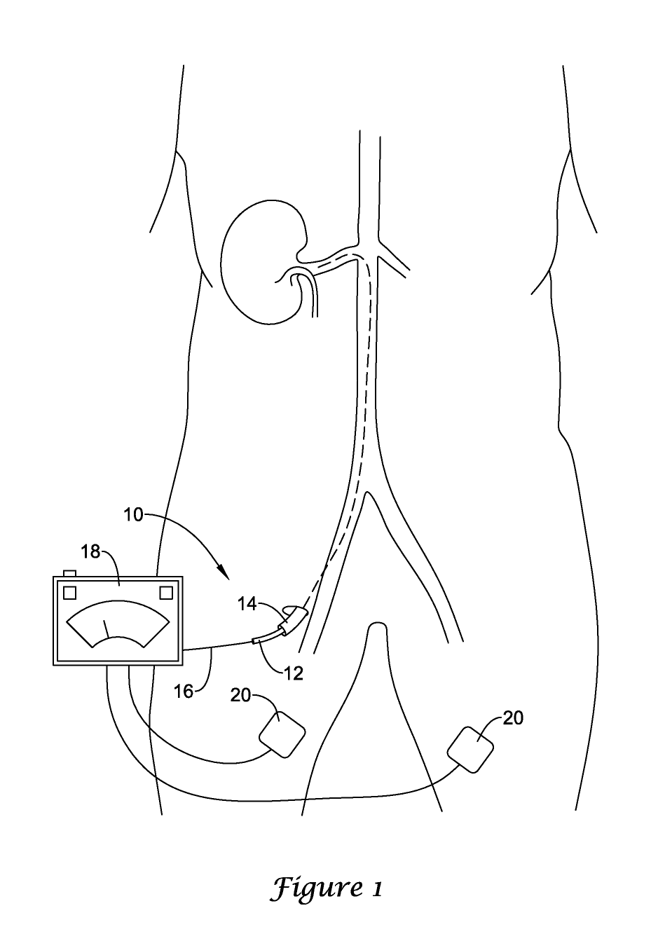

FIG. 1 is a schematic view of an illustrative renal nerve modulation system 10 in situ. System 10 includes a device 12 that includes one or more conductors 16 for providing power to one ore more electrodes (illustrated in subsequent figures) disposed within or on the device 12. The system 10 may include other elements such as a delivery sheath or catheter 14. A proximal end of conductor(s) 16 may be connected to a control and power element 18, which supplies the necessary electrical energy to activate the one or more electrodes in the distal end region of the device 12. One or more return electrode patches 20 may be supplied on the patient's back or at another convenient location on the patient's body to complete the circuit. The control and power element 18 may include monitoring elements to monitor parameters such as power, temperature, voltage, pulse size and/or shape and other suitable parameters as well as suitable controls for performing the desired procedure. In some instances, the power element 18 may control a radio frequency (RF) electrode. The electrode may be configured to operate at a frequency of approximately 460 kHz. It is contemplated that any desired frequency in the RF range may be used, for example, from 450-500 kHz. Lower or higher frequencies may be used, such as 10 kHz or 1000 kHz, in some cases, although the desired heating depth, catheter size, or electrical effects can limit the choice of frequency. However, it is contemplated that different types of energy outside the RF spectrum may be used as desired, for example, but not limited to ultrasound, microwave, and laser.

Some embodiments pertain to the optimization of energy delivery through a multiple electrode renal nerve modulation system. Accordingly, many different renal nerve modulation systems may be suitable.

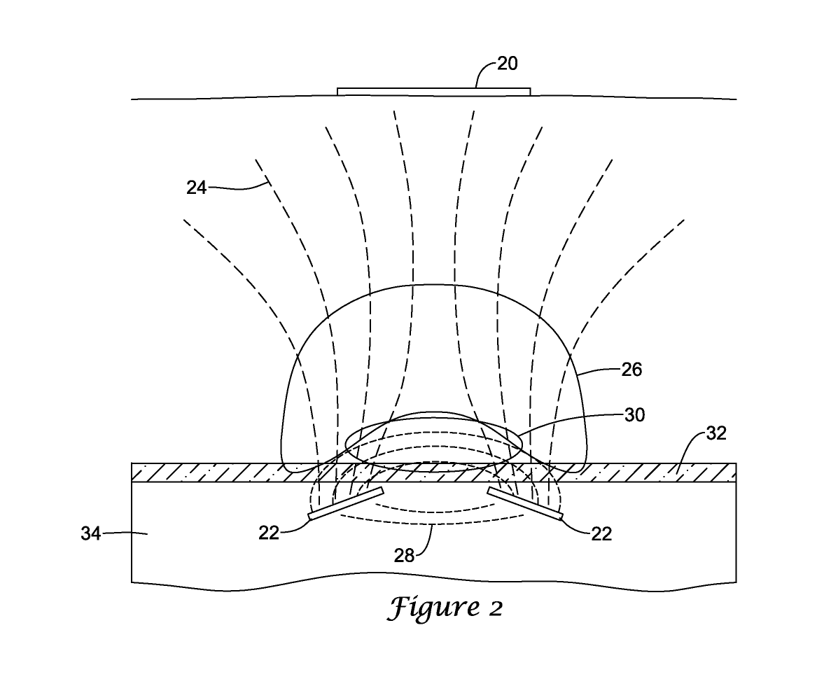

FIG. 2 illustrates, in a highly diagrammatic manner, the distal end of an example embodiment that may be suitable. Only the electrodes 22 of the system illustrated and are depicted in the lumen 34 of a blood vessel. Electrodes 22 are disposed on an elongate member and electrically connected to a control and power system. The distal end region may be expandable through the use of an actuating member or pull wire or may be biased to expand when released from a delivery catheter. The system may include elements such as a spacer cage, a balloon, spacer elements to keep the electrodes from contact with the wall, guide wire lumens and the like.

The two electrodes 22 may be operated in a unipolar mode, where both electrodes are used to transmit the RF energy. In some instances the two electrodes 22 may operate as a single electrode. Broken lines 24, emanating upwards from the electrodes 22 to one or more return electrode patches 20 (see, also, FIG. 1), illustrate the electric fields in a unipolar operation. Such a mode may be suitable for heating tissue in a deeper zone, illustrated at 26. The two electrodes 22 may also be operated in a bipolar mode, where the electrical fields flow between the two electrodes, as indicated at 28. In a bipolar operation, the two electrodes 22 alternately act as the power electrode and the return electrode. Such a mode may be suitable for heating tissue in a shallower zone 30, closer to the artery wall 32. In bipolar mode, the RF current passes from an electrode 22, through the fluid in the blood vessel, the wall 32 of the blood vessel and the body tissue and then back to the control and power unit 18 through another electrode 22. In unipolar mode, the RF current passes from an electrode 22, through the fluid in the blood vessel, the wall 32 of the blood vessel and the body tissue and then back to the control and power unit 18 through a return electrode patch 20 on the outside of the body. Because unipolar and bipolar modes create different denervation patterns, it may be desirable to perform unipolar and bipolar operations during the same procedure.

The control and power system is configured to operate in both the unipolar and bipolar modes. The control and power system may be configured to allow simultaneous unipolar and bipolar operation of the electrodes 22 and the return electrode patch(es) 20 to provide shallow and deep heating a described above. In some embodiments, the control and power system may be configured to periodically alternate operation between unipolar and bipolar operation. In some embodiments, the control and power system is configured to modify the procedure in response to sensor inputs. For example, the system may monitor impedance between individual electrodes 22 and impedance between individual electrodes 22 and the return electrode patch(es) 20 and/or temperature and modify power outputted to one or both of the bipolar and unipolar operations in response to changes in the impedances or the temperature.

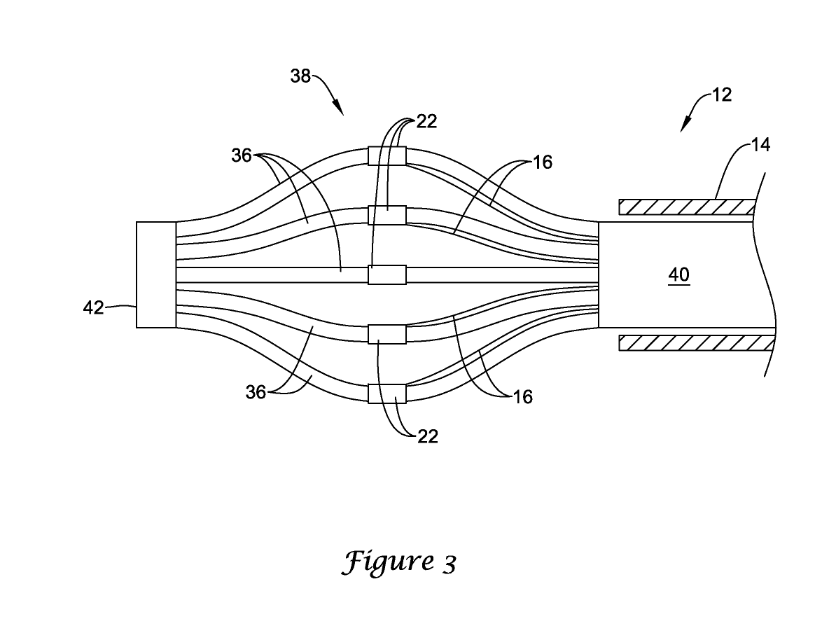

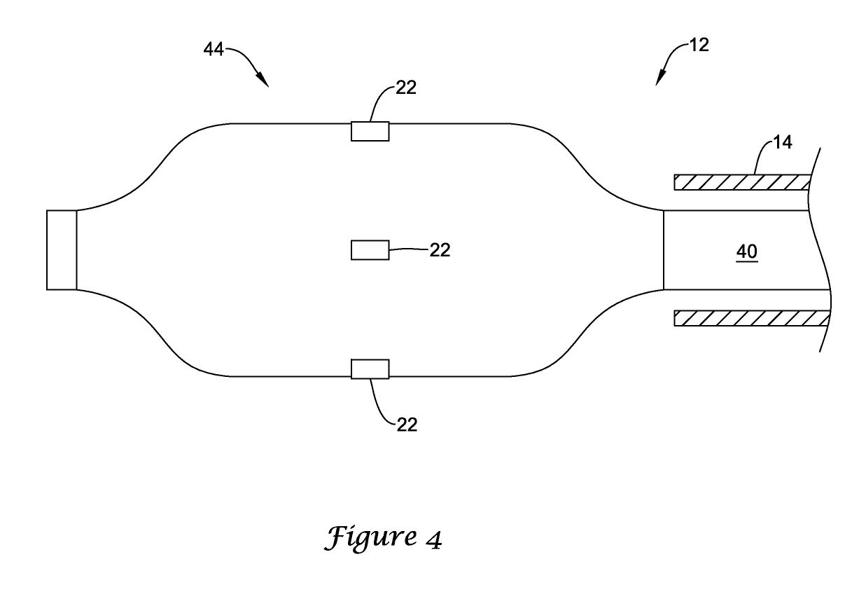

FIGS. 3 and 4 illustrate the distal regions of example systems 10, in which the device 12 is variously an expandable cage assembly 38 comprising a plurality of struts 36, in FIG. 3, or an inflatable balloon 44, as in FIG. 4. Electrodes 22 are disposed on the struts 36 or the balloon 44 as desired. The systems may include other features such as an elongate member or catheter 40, a distal end 42, a guide catheter 14, radiopaque elements, a pull wire, and/or a guide wire and the like. The electrodes 22 may be disposed in a ring at the same longitudinal location or may be disposed in two or more rings at different longitudinal locations. The number of electrodes may vary as desired. For example, there may be 2, 3, 4, 5, 6, 7, 8 or more electrodes in a system. In one example system three electrodes 22 are disposed at one longitudinal location spaced circumferentially from each other and another three electrodes 22 are disposed at a second longitudinal location spaced circumferentially from each other. It can be appreciated that any system that includes more than one electrode at the distal end and an appropriate configured control and power system may be suitable.

In use, any of the systems described herein may be advanced through the vasculature in any manner known in the art. For example, system 10 may include a guidewire lumen to allow the system 10 to be advanced over a previously located guidewire. In some embodiments, the modulation system 10 may be advanced, or partially advanced, within a guide sheath such as the guide catheter 14 shown in FIG. 1. One or more return electrode patches may be placed on the surface of the body at a conventional location. Once the distal end region 27 is placed adjacent to a desired treatment area, the guide catheter 14 may be at least partially withdrawn to expose the distal end region 27. A deflection member may be actuated to position the distal end region near a treatment site. The electrodes 22 may be activated to provide RF energy in both unipolar and bipolar modes. The RF energy may be delivered simultaneously through unipolar and bipolar modes or may be delivered sequentially or alternately as described above. Nerve tissue in the media may be heated by the RF energy and denatured or ablated. The energy profile may be modified during the procedure. Once a particular spot has been treated, the distal end region 27 of the catheter 40 may be moved to treat a second location. For example, the distal end region 27 may be rotated and/or deflected to treat a second location on the same circumferential region of the vessel wall or may be rotated and withdrawn proximally to treat a second location on a different circumferential region of the vessel wall spaced longitudinally and circumferentially from the first treated location. This procedure may be repeated until a desired number of locations have been treated. In some instances, it will be desirable to treat a vessel wall such that the complete circumference of a vessel wall is treated. This circumferential coverage may be provided by treating regions that are spaced longitudinally from each other and are at different circumferential locations or may be provided by treating a complete circumferential ring of the vessel wall.

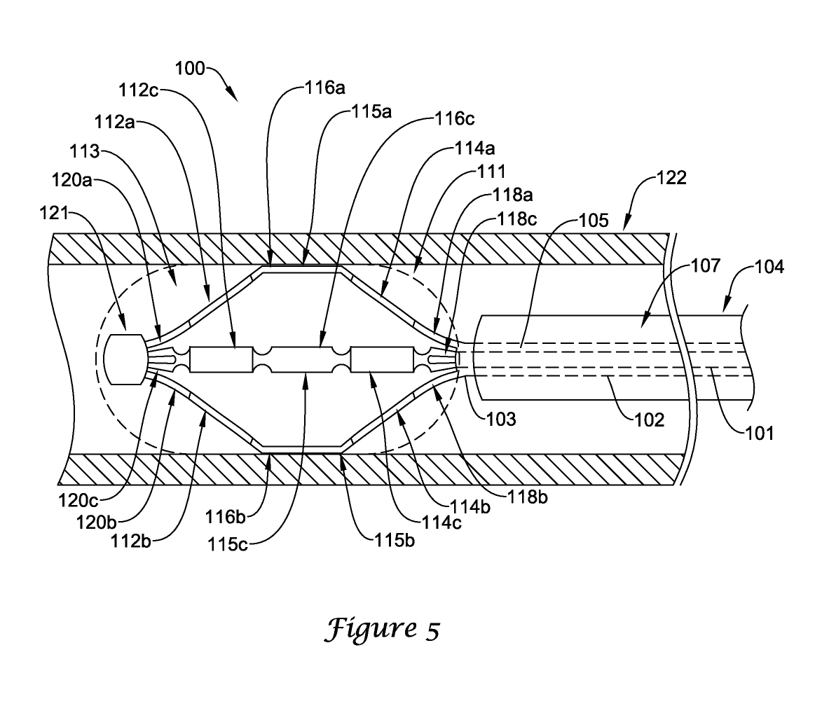

FIG. 5 shows a schematic view of another illustrative renal nerve modulation system 100. As shown, the modulation system 100 may include an elongate shaft 102 having a distal end region 103. The elongate shaft 102 may extend proximally from the distal end region 103 to a proximal end region (not shown) configured to remain outside of a patient's body. In addition, the elongate shaft 102 may include a lumen 107 extending between the distal end region 103 and the proximal end region. While not explicitly shown, the modulation system 100 may further include temperature sensor/wire, an infusion lumen, radiopaque marker bands, a guidewire lumen, and/or other components to facilitate the use and advancement of the system 100 within the vasculature.

In addition, system 100 may include an actuation element such as, for example, an actuation wire 101, which may be slidably disposed within the lumen 105 of the elongate shaft 102. The distal end of the actuation wire 101 may connect to an ablation device 111. In certain instances, the system 100 may further include a sheath, such as a delivery sheath 104 having a proximal end, a distal end, and a lumen 107 extending therebetween such that the elongate shaft 102 including the actuation element 101 may be slidably disposed within the lumen of the delivery sheath 104.

In one embodiment, the actuation wire 101 may be formed from a conductive material covered by an insulating material, however this is not required. It is contemplated that the actuation wire may be formed from other suitable materials. If so provided, the proximal end of the conductive material may be connected to a control unit (such as control unit 18 shown in FIG. 1), which may include an external power generator or battery. The actuation element 101 may transmit an electrical current from the control unit to the ablation device 111 attached to the distal end of the conductor. In addition, or alternatively, the system 100 may include one or more electrical conductors (not explicitly shown), which may attach to the control unit at their proximal ends (not shown) and to the ablation device 111 at their distal ends (not shown) such that the electrical conductors may supply electrical current to the ablation device 111.

In some embodiments, the ablation device 111 may include an expandable frame 113 adapted to transition between collapsed and expanded states. The expandable frame 113 may include a number of expandable positioning elements such as longitudinally extending struts 115A, 115B, and 115C (collectively 115), which may be joined at their proximal and/or distal ends. A person skilled in the art will appreciate that other suitable expandable positioning elements such as, but not limited to, rods or bars, a single hypotube having portions removed to form struts, an expandable stent having the proximal end gathered together, or the like may also be utilized. FIG. 5 illustrates three struts 115 forming the expandable frame 113, though it is contemplated that expandable frame 113 may include any number of struts 115 desired, such as, but not limited to one, two, three, four, five, six, or more.

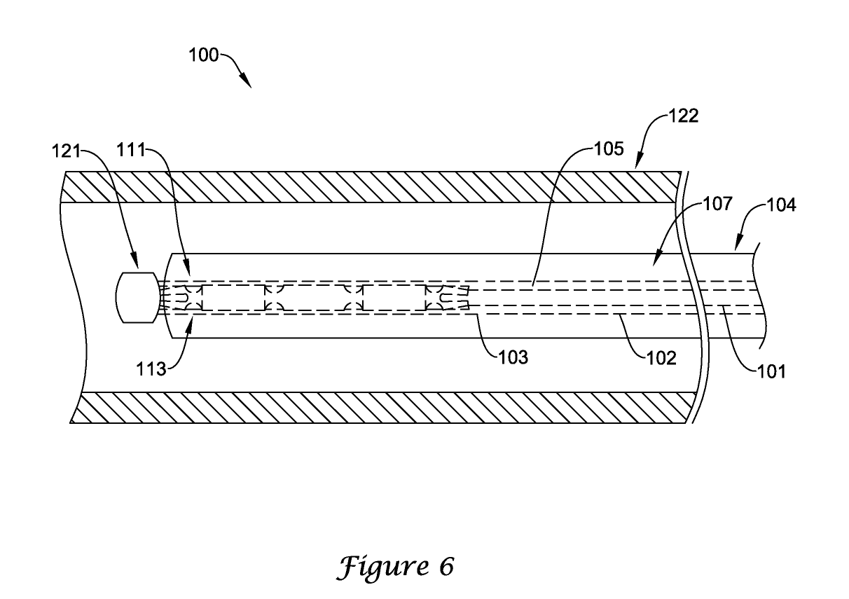

In some instances, the ablation device 111 may be configured to transition between an expanded state (shown in FIG. 5) and a collapsed state (shown in FIG. 6). The ablation device 111 may be self-expandable or may require external force to expand from or be maintained in a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the ablation device 111 may be heat set in the expanded state and then compressed to fit within delivery sheath 104, for example. In another embodiment, a spring may be provided to effect expansion. Alternatively, external forces such as, but not limited to, pneumatic methods, compressed fluid, pull wires, push wires, or the like may also be employed to expand the ablation device 111.

In other instances, a manual force applied to the actuation element 101 may manipulate or actuate the ablation device 111 between the expanded and collapsed state. For example, actuation element may include a central wire that extends through the ablation device 111. According to this embodiment, a pulling force exerted proximally on the wire may allow the struts 115 to expand and move the ablation device 111 into an expanded state. A pushing force exerted distally on the wire may elongate the struts 115 and/or otherwise shift the ablation device to a compressed or elongated state. Other actuation mechanisms may also be utilized.

FIG. 5 depicts the expanded state of the expandable frame 113. As discussed above, the expandable frame 113 may include a number of expandable positioning elements such as longitudinally extending struts 115. The struts 115 may each include a proximal end region 118A, 118B, 118C (collectively 118), an intermediate region 116A, 116B, 116C (collectively 116), and a distal end region 120A, 120B, 120C (collectively 120). It is contemplated that in the expanded state, the intermediate regions 116 of the struts 115 may contact the vessel wall 122. It can be noted that the structure resulting from the expansion of expandable frame 113 positions ablation device 111 for operation. Such a configuration may generally center the ablation device within the vessel and/or maintain a consistent position of the ablation device 111 during the procedure. Those skilled in the art would appreciate that the expandable frame 113 may assume a variety of suitable shapes such as, but not limited to, basket, balloon, or the like without departing from the scope of the disclosure. It is further contemplated that in some embodiments, the struts 115 may have a generally curved or rounded shape in the expanded form.

Struts 115 may be configured to extend generally along the longitudinal axis of the elongate shaft 102. Proximal ends 118A, 118B, and 118C (collectively, 118) of the individual struts 115, which may attach to the distal end 103 of the elongate shaft 102. Further, distal ends 120A, 120B, and 120C (collectively, 120) of struts 115 may attach to a cap 121. In some instances, the cap 121 may include spacers which be used to maintain a consistent spacing between each of the struts 115A, 115B, and 115C. In alternate embodiments, the proximal and distal ends 118, 120 may include a hinge or other similar structures known to those skilled in the art. It is further contemplated that the open structure of the expandable frame 113 may allow blood to flow through the expandable frame 113 for cooling the structure and the vessel wall 122. Therefore, the ablation device 111 may minimize blood stasis, reduce thrombosis, and provide renal perfusion.

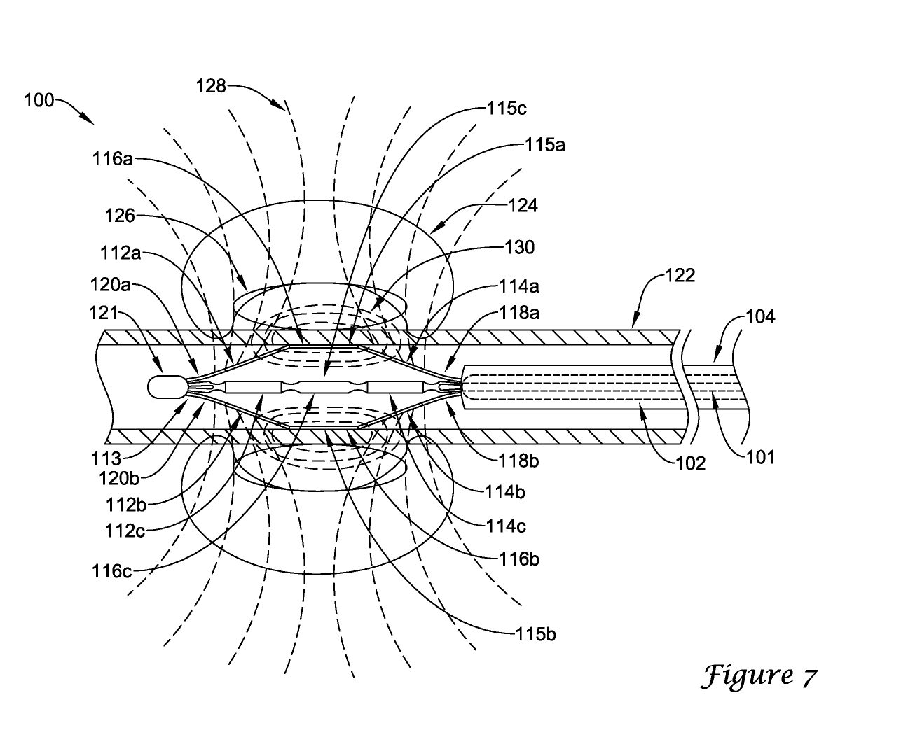

The struts 115 may each include nerve modulation elements such as one or more electrically conductive regions or electrodes 112A, 112B, 112C (collectively 112) positioned adjacent to the distal end regions 120A, 120B, 120C and one or more electrodes 114A, 114B, 114C (collectively 114) positioned adjacent to the distal end regions 120A, 120B, 120C. Alternatively, the electrodes 112, 114 may be placed anywhere along the length of the strut 115 without departing from the scope and spirit of the present disclosure. The illustrated embodiment includes two electrodes per strut (for example, electrodes 112A, 114A disposed on strut 115A), though it is contemplated that the modulation system 100 may include any number of electrodes 112, 114 per strut 115 as desired, such as, but not limited to, one, two, three, four, or more. In addition, electrodes 112A, 112B, and 112C may form a first set of electrodes, whereas electrodes 114A, 114B, and 114C may form a second set of electrodes, as described in connection with FIG. 7 below. The first and second sets of electrodes may include three electrodes 112, 114 each, as shown. In other embodiments, it is contemplated that the first and second set of electrodes may each include any desired number of electrodes, such as, but not limited to, one, two, three, four, or more. In some instances, the number of electrodes in each set may correspond to the number of struts 115.

In addition, the expandable frame 113 may be made of an electrically conductive material, such as, but not limited to, nitinol. In a first instance, the entire frame 113 may be coated with an insulating material, with discrete areas of insulation later removed to form electrically active regions. When so provided, these electrically active regions may define the electrodes. In a second instance, the entire frame 113 may be formed of any material desired and may be coated with an insulating material. Discrete individual electrodes 112, 114 may be affixed to the insulating material of the frame 113 by any suitable means. In a third instance, the expandable frame 113 may include both electrically active regions formed by removing a portion of an insulating material as described above and discrete electrodes affixed to the insulating material. In some embodiments, the electrical current may be directly supplied to the expandable frame 113 via a power and control unit. In other instances, the modulation system 100 may include separate electrical conductors for supplying energy to the electrodes.

It is contemplated that the heating geometry of electrodes 112, 114 may be modified by changing the electrode 112,114 geometry, location and/or spacing. For instance, a single circumferential line of electrodes may be used. Alternatively, the electrodes may employ a staggered geometry. The sets of electrodes 112, 114 may assume a rod-shaped configuration (for example, the electrodes 112, 114 may extend around the entire outer perimeter of the struts 115), however, other suitable shapes of electrodes 112, 114 such as, round, flat, irregular, ovular, or the like may also be contemplated. In some embodiments, the sets of electrodes 112, 114 may employ a broad flat geometry, which may provide increased surface area, and thus may reduce thermal blood damage and fouling of the electrodes, while providing increased flexibility in other segments of the struts 115. In some embodiments, the electrodes 112, 114 may be located/positioned on an exterior surface of the struts 115 (e.g. pointing towards the vessel wall 122). In other embodiments, the electrodes 112, 114 may be located and/or positioned on an interior surface of the struts 115 (e.g. pointing away from the vessel wall 122). In some embodiments, the struts 115 may have one or more electrodes positioned on an interior surface of the intermediate region 116 of the struts 115 that contacts the vessel wall 122. This may position the electrode closer to the desired treatment region without the electrode contacting the vessel wall. However, it is contemplated that in some instances, an electrode may be positioned on the struts 115 such that the electrode contacts the vessel wall 122.

The electrodes 112, 114 may be coupled to a power and control unit, which may provide electrical current to the electrodes 112, 114. As discussed previously, the electrodes 112, 114 may be electrically connected to the power and control unit through the expandable frame 113 or the system 100 may include one or more electrical conductors (for example, wire), which may electrically couple the electrodes 112, 114 to the power and control unit. In certain instances, a single electrical conductor may couple the electrodes 112, 114 to the power and control unit. In other instances, the electrodes 112, 114 may each be individually connected to the power and control unit. It is further contemplated that the electrodes 112, 114 may be electrically connected to the power and control unit as sets (e.g. a first set 112 and a second set 114). It is contemplated that either set of electrodes 112 and 114 may be configured to function in a unipolar mode, a bipolar mode, or both in combination or alone, as described above and in conjunction with FIG. 7 below.

In some embodiments, the electrodes 112, 114 may be positioned on the portions of the ablation device 111 that remain at a distance from the wall 122. For example, the first set of electrodes 112 may be positioned between the distal end regions 120 and the intermediate regions 116. The second set of electrodes 114 may be positioned between the proximal end regions 118 and the intermediate regions 116. Depending on the desired application, electrodes 112, 114 may be placed along different portions of the ablation device 111. For example, in one instance, the electrodes 112, 114 may be placed slightly away from the wall 122. Alternatively, the electrodes 112, 114 may be placed such that they are centered in the vessel (not shown). Here, the electrodes 112, 114 may employ RF-energy to heat or ablate the surrounding target location. Other electrodes employing laser, microwave, or other suitable current sources known to those skilled in the art may also be contemplated. In addition, the electrodes 112, 114 may be spaced from the arterial wall 122, which may avoid tissue injury to the arterial wall 122.

Further, the intermediate regions 116 of the struts 115 may include an insulative element such as a wall-contact alignment region. In one embodiment, the electrodes 112 and 114 may be positioned on proximal and distal ends of intermediate region 116, which may provide an electrical break between the sets of electrodes 112, 114. In that manner, each set of electrodes 112, 114 may be electrically isolated from one another. Suitable examples of wall-contact alignment region 116 capable of providing an electrical separation may include polymers, nonconductive structures, electrically isolated structures, insulated joints, and other suitable structures known to those skilled in the art.

In certain instances, the wall-contact alignment region 116 may have an increased surface area as compared to the set of electrodes 112, 114, and that characteristic may reduce stresses on the artery wall 122 by distributing the force over a larger surface area. Alternatively, the wall-contact alignment region 116 may have a smaller surface area when required. In addition, as discussed above at least a portion of wall-contact alignment region 116 may contact the artery wall 122 when the ablation device 111 assumes the expanded state (shown in FIG. 5).

FIG. 6 shows the collapsed state of the ablation device 111 according to some embodiments of the present disclosure. Here, the expandable frame 113 may lie within the lumen 107 of the delivery sheath 104. As discussed above, many techniques may be utilized to apply sufficient force to the expandable frame 113 to hold it in the collapsed state. According to one technique, the expandable frame 113 is carried within the delivery sheath 104 for deployment. The user may proximally retract the delivery sheath 104 to allow the expandable frame 113 to self-expand. It will be understood that the material and thickness of the delivery sheath 104 may be selected to provide sufficient strength to resist the outward force exerted by expandable frame 113 while still presenting a sufficiently thin profile to allow passage through the vasculature path, without causing injury to the vessel walls 122.

According to another technique, pull wires (such as actuation wire 101) may be utilized to expand the ablation device 111. Pull wires may be attached to either the distal or proximal end of ablation device 111, and by pulling the wire axially (distally or proximally), the operator places a tensile force on the ablation device 111, extending it longitudinally while keeping it in the collapsed state. When the pull wire is released, the ablation device 111 may expand (FIG. 5). For example, if the pull wire is attached to the distal end of ablation device 111, pulling the wire distally elongates (compresses) the ablation device 111 and releasing the pull wire releases the force on the ablation device 111, expanding it. Moreover, an appropriate mechanism to pull, push, or release the pull wire may be configured in a handle (not shown), provided at the operator's end, allowing operators to easily expand or collapse the ablation device 111, as required. Alternatively, the actuation means may be present at the proximal end of the elongate shaft 102.

The expansion of the ablation device 111 should avoid causing damage to the artery by exerting a large force on the artery wall 122. To prevent such problems, the ablation device 111 may include visualization devices such as radiopaque markers or bands, cameras, or fluorescent dyes to visualize the extent of expansion. Further, the ablation device 111 may include a force or expansion-limiting component that prevents the member from expanding beyond a certain limit. In some instances, the expansion limit may be set during manufacturing of the member.

FIG. 7 illustrates a method of using the renal nerve modulation system 100 according to some embodiments of the present disclosure. As discussed above, the modulation system 100 may be operated in various modes. In one embodiment, the system 100 may be operated in a unipolar ablation mode, in which each of the electrodes 112, 114 may be connected to a power supply through power and control unit such that the current may be passed to each electrode separately or simultaneously. In addition, a ground pad (such as ground pad 20 shown in FIG. 1) may be employed, which may be attached to an exterior portion of a patient's body such as, but not limited to, a patient's leg. When the system is operated in a unipolar ablation mode, current 128 may travel between electrodes 112, 114 and the ground pad. In this instance, the electrodes 112, 114 may provide heating to a deeper (e.g. further from the vessel wall) target region 124.

In addition, the unipolar mode may be carried out in two different manners—sequential unipolar mode and simultaneous unipolar mode. In one embodiment, the system 100 may be operated in a sequential unipolar ablation mode. In this mode, the electrodes 112, 114 may each be connected to an independent power supply such that each electrode 112, 114 may be operated separately and current may be maintained to each electrode. In sequential unipolar ablation, one electrode may be activated at a time. The next electrode may be activated only after a first electrode is activated and deactivated. In another instance, the system 100 may operate in a simultaneous unipolar mode, with electrodes 112, 114 activated simultaneously. In this mode, more current may be dispersed radially as all the electrodes collectively emanate current at the same time. This dispersion may result in a more effective, deeper penetration compared to the sequential unipolar mode.

In another instance, the system 100 may operate in a bipolar mode. In this mode, the sets of electrodes 112, 114 disposed at the treatment location may be 180° out of phase such that one electrode acts as the ground electrode (e.g. one cathode and one anode). As such current 130 may flow around the ablative member from proximal electrodes 112 to the distal electrodes 114. Bipolar mode may provide shallower heating to a target region 126 adjacent to the vessel wall 122 than unipolar mode. In general, the unipolar mode may penetrate more deeply than the bipolar mode, therefore providing ablation to a wider range of nerve tissues. Any of the embodiments described in this disclosure may be operated in any of the above-described modes.

In certain instances, the unipolar and bipolar modes may be modulated by cycling between them over time, and duty cycle and/or power levels may be varied. Alternatively, unipolar and bipolar modes may be activated simultaneously, with current 130 between one set of electrodes 112 and another set of electrodes 114, and current 128 between one set of electrodes 112, 114 and a remote ground pad 110. In addition, more sets of electrodes may be incorporated to enable the desired unipolar and bipolar activations and heating pattern.

In use, the system 100 may be introduced percutaneously using conventional methods. For example, a guidewire may be introduced percutaneously and navigated to a target location using standard radiographic techniques. This is just an example. Optionally, a guide catheter (not explicitly shown) may be introduced over the guide wire and the guide wire may be withdrawn. The delivery sheath 104, elongate shaft 102, and the ablation device 111 may then be introduced together within a lumen of the guide catheter and urged distally to the desired location. Once there, the guide catheter and/or delivery sheath 104 may be retracted proximally and the actuation element 101 may be manipulated to allow the ablation device 111 to expand in any of the manners discussed above.

The electrodes 112, 114 may then be activated to ablate and/or modulate target tissue. It is contemplated that the electrodes 112, 114 may be activated in a unipolar or bipolar mode, or a combination thereof, as desired. During this procedure, the ablation device 111 may continuously monitor the temperature at the electrodes 112, 114 and the vessel wall 122. Radiography techniques may be utilized to monitor the tissue being ablated. Other monitoring methods may also be utilized. Once the tissue is sufficiently ablated, the ablation device 111 may be retracted to the collapsed state (shown in FIG. 6) and retrieve it from the patient's body. As desired, the ablation device 111 may be longitudinally repositioned and reactivated to target a longer length of target tissue.

Further, to monitor the temperature of the electrodes 112, 114 and the vessel wall 122, one or more sensors (not shown), such as temperature sensors, may be placed at different portions of the ablation device 111. For instance, one sensor may be placed near the electrodes 112, 114 to monitor electrode fouling or electrode temperature and another sensor may be placed in the portion contacting the vessel wall 122 to measure the temperature of the blood vessel. The sensors may be configured to provide feedback to the power and control unit for adjusting parameters such as, but not limited to, power, voltage, current, duty cycle, duration, and so forth. In addition, the power and control unit may be configured to raise alerts if any of the sensors detect temperatures over a preconfigured threshold value. If an alert is raised, operators may discontinue modulation until the temperature at the electrode 112, 114 or at the artery wall 122 falls below the threshold value. Alternatively, operators may simply monitor the temperatures and discontinue when temperatures exceed a certain value. In general, impedance of surrounding tissue may be measured as an indication of heating and ablation. Temperature and/or impedance measurements may also be utilized to adjust a treatment regimen and/or to otherwise determine whether to utilize system 100 in a unipolar mode, in a bipolar mode, or both.

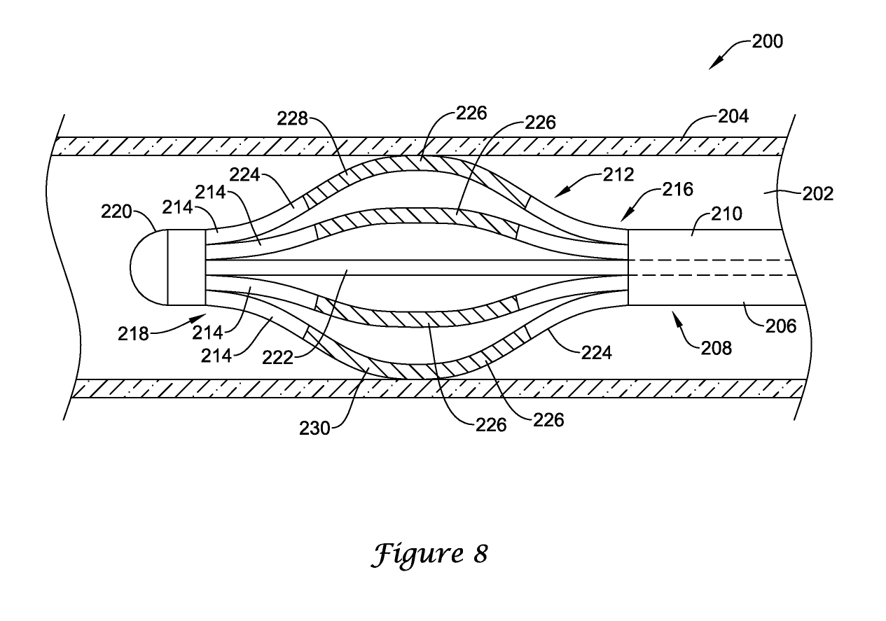

FIG. 8 shows a side view of another illustrative renal nerve modulation system 200 disposed within a body lumen 202 having a vessel wall 204. The system 200 may include an elongate catheter shaft 206 having a proximal end (not shown) and a distal end region 208. The elongate shaft 206 may extend proximally from the distal end region 208 to the proximal end configured to remain outside of a patient's body. Although not shown, the proximal end of the elongate shaft 206 may include a hub attached thereto for connecting other treatment devices or providing a port for facilitating other treatments. It is contemplated that the stiffness of the elongate shaft 206 may be modified to form the modulation system 200 for use in various vessel diameters and various locations within the vascular tree.

In some instances, the elongate shaft 206 may have an elongate tubular structure and may include one or more lumens 210 extending therethrough. In some embodiments, the elongate shaft 206 may include one or more guidewire or auxiliary lumens. In some instances, the elongate shaft 206 may include a separate lumen(s) (not shown) for infusion of fluids, such as saline or dye for visualization or for other purposes such as the introduction of a medical device, and so forth. The fluid may facilitate cooling of the modulation system 200 during the ablation procedure, in addition to the cooling of a body lumen. Further, the lumens may be configured in any way known in the art. For example, the lumen(s) 210 may extend along the entire length of the elongate shaft 206 such as in an over-the-wire catheter or may extend only along a distal portion of the elongate shaft 206 such as in a single operator exchange (SOE) catheter. These examples are not intended to be limiting, but rather examples of some possible configurations. While not explicitly shown, the modulation system 200 may further include temperature sensors/wire, an infusion lumen, radiopaque marker bands, fixed guidewire tip, a guidewire lumen, and/or other components to facilitate the use and advancement of the system 200 within the vasculature.

Further, the elongate shaft 206 may have a relatively long, thin, flexible tubular configuration. In some instances, the elongate shaft 206 may have a generally circular cross-section, however, other suitable configurations such as, but not limited to, rectangular, oval, irregular, or the like may also be contemplated. In addition, the elongate shaft 206 may have a cross-sectional configuration adapted to be received in a desired vessel, such as a renal artery. For instance, elongate shaft 206 may be sized and configured to accommodate passage through an intravascular path, which leads from a percutaneous access site in, for example, the femoral, brachial, or radial artery, to a targeted treatment site, for example, within a renal artery.

The modulation system 200 may further include an expandable basket 212 positioned adjacent the distal end region 208 of the elongate shaft 206. The basket 212 may be configured to move between a collapsed position (not explicitly shown) and an expanded position, as shown in FIG. 8. The proximal end 216 of the expandable basket 212 may be affixed to the elongate shaft 206 adjacent to the distal end region 208 and the distal end 218 may be affixed to an end cap 220. In some instances, the cap 220 may include spacers which be used to maintain a consistent spacing between each of the struts 214. The proximal and distal ends 216, 218 of the basket 212 may be affixed to the elongate shaft 206 and/or cap 220 in any manner desired. For example, in some instances, a band or retaining element may be used to secure the proximal and distal ends 216, 218. In other instances, the proximal and distal ends 216, 218 may be secured to the elongate shaft 206 and/or cap 220 with an adhesive or other suitable method.

The basket 212 may include a plurality of ribbons, tines, or struts 214 extending from a proximal end 216 to a distal end 218 of the basket 212. Although four struts 214 are shown in FIG. 8, it should be noted that any suitable number of struts 214 may be employed for a desired purpose. It is also contemplated that the struts 214 may have any cross-sectional shaped desired, such as, but not limited to, circular, square, rectangular, oval, polygonal, etc. Further, the expandable basket 212 may be configured to actuate between a first collapsed configuration and a second expanded configuration (shown in FIG. 8), which may include transition of the struts 214 from a generally straight configuration to a curved configuration, respectively. More particularly, the struts 214 in the collapsed configuration may extend and/or straighten to be generally parallel with or generally extend along the longitudinal length of the elongate shaft 206. In contrast, in the second expanded configuration, as shown in FIG. 8, the struts 214 may expand and/or curve like the ribs of an umbrella to contact the vessel wall 204.

The basket 212 may be self-expandable or may require external force to expand from or be maintained in a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the basket 212 may be heat set in the expanded state and then compressed to fit within delivery sheath, for example. In another embodiment, a spring may be provided to effect expansion. Alternatively, external forces such as, but not limited to, pneumatic methods, compressed fluid, pull wires, push wires, or the like may also be employed to expand the basket 212.

In other instances, a manual force applied to a control wire 222 may manipulate or actuate the basket 212 between the expanded and collapsed state. For example, control wire 222 may include a central wire that extends through the basket 212 and the elongate shaft 206. In some embodiments, a distal end of the control wire 222 may be fixedly secured to the end cap 220 or to the distal end 218 of the basket 212 and extend proximally to a location configured to remain outside the body. According to this embodiment, a pushing or pulling force exerted on the wire may allow the struts 214 to expand and move the basket 212 into an expanded state. A pushing or pulling force exerted on the wire may elongate the struts 214 and/or otherwise shift the basket 212 to a compressed or elongated state. Other actuation mechanisms may also be utilized. In some embodiments, the control wire 222 may be formed from a conductive material and may be used to supply electrical energy to the basket 212. In such an instance, the proximal end of the control wire 222 may be connected to a control unit (such as control unit 18 shown in FIG. 1), which may include an external power generator or battery. The control wire 222 may transmit an electrical current from the control unit to the basket 212 attached to a distal end of the control wire 222. In addition, or alternatively, the system 200 may include one or more electrical conductors (not explicitly shown), which may attach to the control unit at their proximal ends (not shown) and to the basket 212 at their distal ends (not shown) such that the electrical conductors may supply electrical current to the basket 212.

In some embodiments, the expandable basket 212 may be formed from a conductive material covered with an insulating layer 224. The expandable basket 212 may be coated with insulating material using any number of coating techniques, such as, but not limited to, dip coating, spray coating, etc. In some instances, the expandable basket 212 may be coated with parylene or other insulating material. In other instances, an insulating tube, such as polyethylene terephthalate (PET), perfluoroalkoxy (PFA), or other insulator, may be slid onto each strut 214. It is contemplated that the insulating layer 224 may be removed from or not applied to one or more locations on the expandable basket 212 to form one or more electrically conductive regions 226 configured to deliver RF energy to the target region around the vessel wall 204. In some embodiments, masking techniques may be used to create electrically conductive regions 226. It is contemplated that the insulating layer 224 may be absent from the entire perimeter of the strut 214 or from only selected portions of the perimeter, as desired. The one or more electrically conductive regions 226 may function as one or more electrodes for delivering RF energy to a desired treatment area. In the expanded configuration, one or more electrically conductive regions 226 may contact the vessel wall 204 along some portions and be spaced from the vessel wall 204 along other portions, as shown in FIG. 8. The energy delivery regions 226 may be positioned on the struts 214 such that the energy delivery regions 226 are spaced about the circumference of the lumen 202. While each strut 214 is illustrated as include a single energy delivery regions 226, it is contemplated that any number of energy delivery regions 226 may be provided on any of the struts 214, as desired. Various basket 212 and strut 214 configurations can be used, and the energy delivery regions 226 can be arranged to optimize the ablation regions, as desired.

It is contemplated that the modulation system 200 may be advanced through the vasculature to a desired treatment region, such as the renal artery. The modulation system 200 may be advanced with the expandable basket 212 in a collapsed position. In some instance, a delivery sheath or guide catheter may be used to facilitate advancement of the system 200. When the expandable basket 212 is positioned adjacent to the target treatment region, the control wire 222 may be actuated to expand the basket 212. In the expanded configuration, portions of the outer surface of the expandable basket 212, including portions of electrically conductive regions 226, may come into gentle contact with the vessel wall 204.

The strut 214 orientation angle when deployed may affect the position of electrically conductive regions 226 and can thus affect the heating pattern. The current may spread out in the blood before passing through the vessel wall 204 and into the target perivascular tissue. The geometry of heated or ablated region can be affected by the overall length of the electrically conductive regions 226, for example. Configurations with greater length may require higher power to be effective, which can increase the depth of heating in some areas.

One or more electrical conductors (not explicitly shown) may connect the expandable basket 212 to a power and control unit which provides RF energy to the expandable basket 212. Alternatively, power may be supplied to the basket 212 through control wire 222. In some instances, RF energy may be supplied to the entire basket 212, but is only emitted from the electrically conductive regions 226. It is contemplated that the electrically conductive regions 226 may function as multiple electrodes connected in parallel to deliver RF energy to the desired treatment region, however this is not required. In some instances, the electrically conductive regions 226 may be separately powered and controlled. When the electrically conductive regions are powered in parallel, a single-channel control unit may provide power to the electrically conductive regions 226 simultaneously. This may allow for multi-point ablation while reducing procedure time compared to performing sequential ablation of discrete spots. It is further contemplated that simultaneous ablation of multiple treatment locations may also avoid or reduce overlapping treatment areas or widely separated treatment areas. In some instances, overlapping treatment areas may cause locally severe damage to the vessel or other adjacent tissue. Widely separated treatment areas may leave untreated nerves, making the therapy less effective. Some portions 228 of energy delivery regions 226 may be in direct contact with the vessel wall 204, providing effective ablation of nearby nerves. Other portions 230 of the energy delivery regions 226 may be held a controlled distance away from the vessel wall, providing ablation of deeper nerves. The combination of wall-contact 228 and off-wall 230 portions of energy delivery regions 226 may provide lower current densities than other wall-contact approaches which may reduce vessel wall burns, while extending the ablation zone to treat somewhat deeper nerves. The combination of wall-contact 228 and off-wall 230 portions of energy delivery regions 226 may also reduce current densities enough to avoid or reduce blood damage and fouling of the energy delivery region 226 surfaces.

It is contemplated that a ground pad such as ground pads 20 shown in FIG. 1 can be used to complete the circuit, energizing the energy delivery regions 226 in a unipolar manner. Alternatively, the struts 214 can be electrically isolated from each other, and energized between struts 214 in a bipolar manner. In other instances, multiple energy delivery regions 226 may be provided on the same strut 214 with an electrical break provided between the energy delivery regions 226 such that the energy delivery regions 226 can be energized in a bipolar manner along the same strut 214.

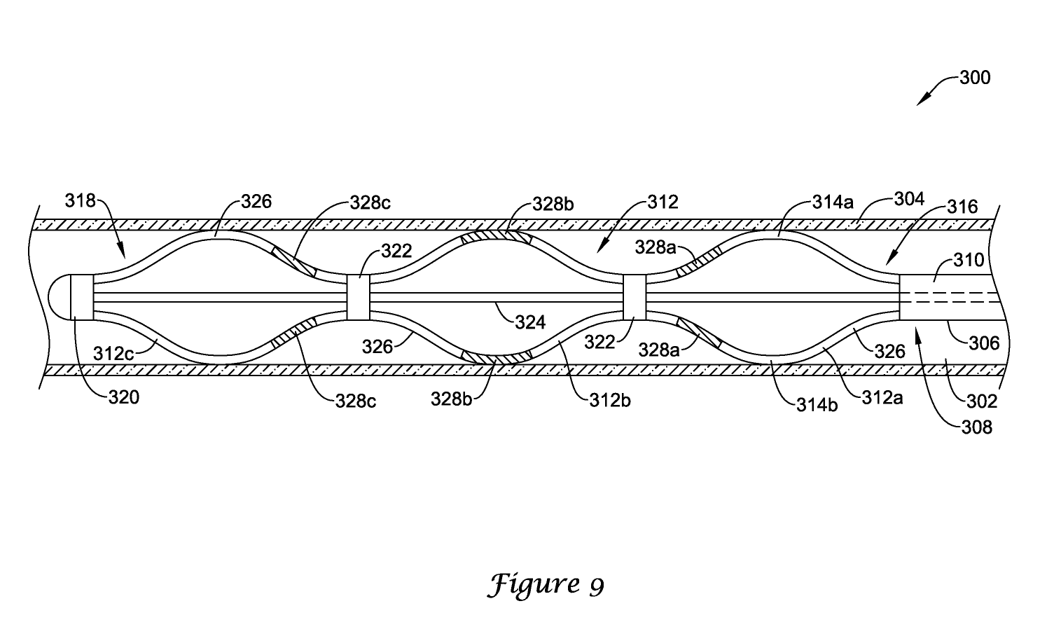

FIG. 9 shows a side view of another illustrative renal nerve modulation system 300 disposed within a body lumen 302 having a vessel wall 304. The system 300 may include an elongate catheter shaft 306 having a proximal end (not shown) and a distal end region 308. The elongate shaft 306 may extend proximally from the distal end region 308 to the proximal end configured to remain outside of a patient's body. Although not shown, the proximal end of the elongate shaft 306 may include a hub attached thereto for connecting other treatment devices or providing a port for facilitating other treatments. It is contemplated that the stiffness of the elongate shaft 306 may be modified to form the modulation system 300 for use in various vessel diameters and various locations within the vascular tree. In some instances, the elongate shaft 306 may have an elongate tubular structure and may include one or more lumens 310 extending therethrough. While not explicitly shown, the modulation system 300 may further include temperature sensors/wire, an infusion lumen, radiopaque marker bands, fixed guidewire tip, a guidewire lumen, and/or other components to facilitate the use and advancement of the system 300 within the vasculature. Elongate shaft 306 may be similar in form and function to elongate shaft 206 discussed above.

The modulation system 300 may further include an expandable basket 312 positioned adjacent the distal end region 308 of the elongate shaft 306. The basket 312 may be configured to move between a collapsed position (not explicitly shown) and an expanded position, as shown in FIG. 9. The proximal end 316 of the expandable basket 312 may be affixed to the elongate shaft 306 adjacent to the distal end region 308310 and the distal end 318 may be affixed to 315 may be affixed to an end cap 220. In some instances, the cap 220 may include spacers which be used to maintain a consistent spacing between each of the struts 314. The proximal and distal ends 316, 318 of the basket 312 may be affixed to the elongate shaft 306 and/or cap 220 in any manner desired. For example, in some instances, a band or retaining element may be used to secure the proximal and distal ends 316, 318. In other instances, the proximal and distal ends 316, 318 may be secured to the elongate shaft 306 and/or cap 220 with an adhesive or other suitable method.

The basket 312 may include one or more ribbons, tines, or struts 314A, 314B (collectively 314) extending from a proximal end 316 to a distal end 318 of the basket 312. Although two struts 314 are shown in FIG. 9, it should be noted that any suitable number of struts 314 may be employed for a desired purpose. It is also contemplated that the struts 314 may have any cross-sectional shaped desired, such as, but not limited to, circular, square, rectangular, oval, polygonal, etc. Further, the expandable basket 312 may be configured to actuate between a first collapsed configuration and a second expanded configuration (shown in FIG. 9), which may include transition of the struts 314 from a generally straight configuration to a curved configuration, respectively. More particularly, the struts 314 in the collapsed configuration may extend and/or straighten to be generally parallel with or generally extend along the longitudinal length of the elongate shaft 306. In contrast, in the second expanded configuration, as shown in FIG. 9, the struts 314 may expand and/or curve like the ribs of an umbrella to contact the vessel wall 304. In some embodiments, the basket 312 may include a plurality of intermediate collars 324 which may maintain portions of the struts 314 in a collapsed position when the basket 312 is expanded effectively creating multiple basket portions 312A, 312B, 312C (collectively 312). It is contemplated that any number of intermediate collars 324 may be used to form the desired structure. In some instances, collars 324 may be tubular elements fixedly or movable secured to the struts 314 at various intervals.

The basket 312 may be self-expandable or may require external force to expand from or be maintained in a collapsed state. Self-expandable members may be formed of any material or structure that is in a compressed state when force is applied and in an expanded state when force is released. Such members may be formed, for example, of shape memory alloys such as nitinol or any other self-expandable materials. When employing such shape-memory materials, the basket 312 may be heat set in the expanded state and then compressed to fit within delivery sheath, for example. In another embodiment, a spring may be provided to effect expansion. Alternatively, external forces such as, but not limited to, pneumatic methods, compressed fluid, pull wires, push wires, or the like may also be employed to expand the basket 312.

In other instances, a manual force applied to a control wire 324 may manipulate or actuate the basket 312 between the expanded and collapsed state. For example, control wire 324 may include a central wire that extends through the basket 312 and the elongate shaft 306. In some embodiments, a distal end of the control wire 324 may be fixedly secured to the end cap 320 or to the distal end 318 of the basket 312 and extend proximally to a location configured to remain outside the body. According to this embodiment, a pushing or pulling force exerted on the wire may allow the struts 314 to expand and move the basket 312 into an expanded state. A pushing or pulling force exerted on the wire may elongate the struts 314 and/or otherwise shift the basket 312 to a compressed or elongated state. Other actuation mechanisms may also be utilized. In some embodiments, the control wire 324 may be formed from a conductive material and may be used to supply electrical energy to the basket 312. In such an instance, the proximal end of the control wire 324 may be connected to a control unit (such as control unit 18 shown in FIG. 1), which may include an external power generator or battery. The control wire 324 may transmit an electrical current from the control unit to the basket 312 attached to a distal end of the control wire 324. In addition, or alternatively, the system 300 may include one or more electrical conductors (not explicitly shown), which may attach to the control unit at their proximal ends (not shown) and to the basket 312 at their distal ends (not shown) such that the electrical conductors may supply electrical current to the basket 312.

In some embodiments, the expandable basket 312 may be formed from a conductive material covered with an insulating layer 326. The expandable basket 312 may be coated with insulating material using any number of coating techniques, such as, but not limited to, dip coating, spray coating, etc. In some instances, the expandable basket 312 may be coated with parylene or other insulating material. In other instances, an insulating tube, such as polyethylene terephthalate (PET), perfluoroalkoxy (PFA), or other insulator, may be slid onto each strut 314. It is contemplated that the insulating layer 326 may be removed from or not applied to one or more locations on the expandable basket 312 to form one or more electrically conductive regions 328A, 328B, 328C (collectively 328) configured to deliver RF energy to the target region around the vessel wall 304. It is contemplated that the insulating layer 326 may be absent from the entire perimeter of the strut 314 or from only selected portions of the perimeter, as desired. The one or more electrically conductive regions 328 may function as one or more electrodes for delivering RF energy to a desired treatment area. In the expanded configuration, one or more electrically conductive regions 328B may contact the vessel wall 304 along some portions and one or more electrically conductive regions 328A, 328C may be spaced from the vessel wall 304, as shown in FIG. 9.

The basket 312 can be symmetric or asymmetric, as desired. For example, some struts 314 can be staggered from other struts 314. The struts 314 can be generally axial, or can have circumferential or spiral orientation. Portions of the basket 312 such as proximal portion 312A, intermediate portion 312B, or distal portion 312C can be of different sizes or the same size as desired. In some embodiments, portions of struts 314 can be wider to increase the surface area of electrically conductive regions 328, for example. The electrically conductive regions 328 can be arranged in a spiral pattern, in a longitudinal line, or random, as desired. The energy delivery regions 328 may be positioned on the struts 314 such energy is delivered in a desired pattern. While each strut 314A, 314B is illustrated as including three energy delivery regions 328A, 328B, 328C, it is contemplated that any number of energy delivery regions 328 may be provided on any of the struts 314, as desired. Various basket 312 and strut 314 configurations can be used, and the energy delivery regions 328 can be arranged to optimize the ablation regions, as desired.

It is contemplated that the modulation system 300 may be advanced through the vasculature to a desired treatment region, such as the renal artery. The modulation system 300 may be advanced with the expandable basket 312 in a collapsed position. In some instance, a delivery sheath or guide catheter may be used to facilitate advancement of the system 300. When the expandable basket 312 is positioned adjacent to the target treatment region, the control wire 324 may be actuated to expand the basket 312. In the expanded configuration, portions of the outer surface of the expandable basket 312, including electrically conductive regions 328B, may come into gentle contact with the vessel wall 304. Other portions of the basket 312 and electrically conductive regions 328A, 328C may remain spaced a distance from the vessel wall 304.

One or more electrical conductors (not explicitly shown) may connect the expandable basket 312 to a power and control unit which provides RF energy to the expandable basket 312. Alternatively, power may be supplied to the basket 312 through control wire 324. In some instances, RF energy may be supplied to the entire basket 312, but is only emitted from the electrically conductive regions 328. It is contemplated that the electrically conductive regions 328 may function as multiple electrodes connected in parallel to deliver RF energy to the desired treatment region, however this is not required. In some instances, the electrically conductive regions 328 may be separately powered and controlled. When the electrically conductive regions 328 are powered in parallel, a single-channel control unit may provide power to the electrically conductive regions 328 simultaneously. This may allow for multi-point ablation while reducing procedure time compared to performing sequential ablation of discrete spots. It is further contemplated that simultaneous ablation of multiple treatment locations may also avoid or reduce overlapping treatment areas or widely separated treatment areas. In some instances, overlapping treatment areas may cause locally severe damage to the vessel or other adjacent tissue. Widely separated treatment areas may leave untreated nerves, making the therapy less effective. Some energy delivery regions 328B may be in direct contact with the vessel wall 304, providing effective ablation of nearby nerves. Other energy delivery regions 328A, 328C may be held a controlled distance away from the vessel wall 304, providing ablation of deeper nerves. The combination of wall-contact 328B and non-wall contact 328A, 328C energy delivery regions 328 may provide lower current densities than other wall-contact approaches which may reduce vessel wall burns, while extending the ablation zone to treat somewhat deeper nerves. The combination of wall-contact 328B and non-wall contact 328A, 328C energy delivery regions 328 may also reduce current densities enough to avoid or reduce blood damage and fouling of the energy delivery region 328 surfaces. Further, the struts 314 may have a large surface to volume ratio thus, the heat transfer to the blood for cooling may be greater than with conventional ellipsoid or cylindrical shaped electrodes.

It is contemplated that a ground pad such as ground pads 20 shown in FIG. 1 can be used to complete the circuit, energizing the energy delivery regions 328 in a unipolar manner. Alternatively, the struts 314 can be electrically isolated from each other, and energized between struts 314 in a bipolar manner. In other instances, multiple energy delivery regions 328 may be provided on the same strut 314 with an electrical break provided between the energy delivery regions 328 such that the energy delivery regions 328 can be energized in a bipolar manner along the same strut 314.

The materials that can be used for the various components of systems 100, 200, 300 (and/or other systems disclosed herein) may include those commonly associated with medical devices. For simplicity purposes, the following discussion makes reference to shaft 102. However, this is not intended to limit the systems and methods described herein, as the discussion may be applied to other components in systems 100, 200, 300.

Shaft 102 and/or other components of systems 100, 200, 300 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of system 100 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of systems 100, 200, 300 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of system 100 to achieve the same result.

In some embodiments, a degree of Magnetic Resonance Imaging (MRI) compatibility is imparted into systems 100, 200, 300. For example, shaft 102 or portions thereof, may be made of a material that does not substantially distort the image and create substantial artifacts (i.e., gaps in the image). Certain ferromagnetic materials, for example, may not be suitable because they may create artifacts in an MRI image. Shaft 102 or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers that may be suitable for use in system 100 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

Although the embodiments described above have been set out in connection with a renal nerve ablation catheter, those of skill in the art will understand that the principles set out there can be applied to any catheter or endoscopic device where it is deemed advantageous to deflect the tip of the device. Conversely, constructional details, including manufacturing techniques and materials, are well within the understanding of those of skill in the art and have not been set out in any detail here. These and other modifications and variations my well within the scope of the present disclosure and can be envisioned and implemented by those of skill in the art.

Those skilled in the art will recognize that the present invention may be manifested in a variety of forms other than the specific embodiments described and contemplated herein. Accordingly, departure in form and detail may be made without departing from the scope and spirit of the present invention as described in the appended claims.

What is claimed is:

1. A nerve modulation system comprising:
an elongate shaft having a proximal end and a distal end;
an expandable member coupled to the elongate shaft adjacent to the distal end of the shaft;
a plurality of electrically conductive regions disposed on the expandable member for emitting an electrical current, the plurality of electrically conductive regions comprising at least a first electrically conductive region and a second electrically conductive region;
a ground pad; and
a control unit electrically coupled to the plurality of electrically conductive regions and the ground pad, wherein the control unit is capable of operating in a unipolar mode and a bipolar mode during a single procedure.

2. The nerve modulation system of claim 1, wherein the expandable member comprises an inflatable balloon.

3. The nerve modulation system of claim 1, wherein the expandable member comprises an expandable basket including a plurality of longitudinally extending struts.

4. The nerve modulation system of claim 3, wherein the first electrically conductive region is positioned on a first strut of the plurality of struts and the second electrically conductive region is positioned on a second strut of the plurality of struts.

5. The nerve modulation system of claim 3, wherein the first and second electrically conductive regions are positioned on a first strut of the plurality of struts.

6. The nerve modulation system of claim 3, wherein at least some of the plurality of longitudinally extending struts are capable of partially contacting a vessel wall when the expandable member is in an expanded state.

7. The nerve modulation system of claim 6, wherein at least a portion of at least one of the plurality of electrically conductive regions is capable of contacting a vessel wall when the expandable member is in the expanded state.

8. The nerve modulation system of claim 1, wherein the first electrically conductive region and the second electrically conductive region are operated in a unipolar mode such that current flows between the first and/or second electrically conductive regions and the ground pad.

9. The nerve modulation system of claim 1, wherein the first and second electrically conductive regions are operated in a bipolar mode such that current flows between the first and second electrically conductive regions.

10. The nerve modulation system of claim 1, wherein the system is capable of alternating between operating in a unipolar mode and operating in a bipolar mode.

11. A system for nerve modulation comprising:
an elongate shaft having a proximal end region, a distal end region, and a lumen disposed therebetween;
an actuation element slidably disposed within the lumen of the elongate shaft;
an expandable frame having a proximal end and a distal end, the expandable frame coupled to a distal end region of the actuation element;
a first set of electrodes disposed adjacent the proximal end of the expandable frame; and
a second set of electrodes disposed adjacent the distal end of the expandable frame.

12. The nerve modulation system of claim 11, wherein the first set of electrodes comprises two or more electrodes.

13. The nerve modulation system of claim 11, wherein the second set of electrodes comprises two or more electrodes.

14. A system for nerve modulation comprising:
an elongate shaft having a proximal end region, a distal end region, and a lumen disposed therebetween;
an actuation element slidably disposed within the lumen of the elongate shaft;
an expandable basket including two or more struts and having a proximal end and a distal end, the expandable frame coupled to a distal end region of the actuation element;
a return electrode patch;
a first electrically conductive region disposed on a first strut of the two or more struts; and
a second electrically conductive region disposed on a second strut of the two or more struts.

15. The system of claim 14, wherein the plurality of struts comprise an electrically conductive material coated with a non-conductive material and the first and second electrically conductive regions are defined by regions of the struts free from the non-conductive material.

16. The system of claim 14, wherein the first electrically conductive region includes a wall contact portion capable of contacting a vessel wall when the expandable basket is in an expanded configuration and a second non-wall contact portion capable of being spaced a distance from the vessel wall when the expandable basket is in an expanded configuration.

17. The system of claim 14, wherein the expandable basket includes a plurality of basket regions.

18. The system of claim 17 wherein the first electrically conductive region is disposed within a first basket region of the plurality of basket regions and the second electrically conductive region is disposed within a second basket region of the plurality of basket regions.

19. The system of claim 14 further comprising a control and power unit electrically connected to the first and second electrically conductive regions and the return electrode patch, wherein the control and power unit is capable of operating in a unipolar mode and a bipolar mode during the same procedure.

20. The system of claim 19 wherein the control and power unit is capable of switching between unipolar mode and bipolar mode upon receiving user input.
